(19) United States
(12) Patent Application Publication
(10) Pub. No.: US 2026/0191564 A1
(43) Pub. Date: Jul. 9, 2026

(54) EXTERNAL FIXATION SYSTEM, INSTRUMENTATION, AND SURGICAL METHODS

(71) Applicant: Paragon 28, Inc., Englewood, CO (US)

(72) Inventor: Francis HAURIS, Englewood, CO (US)

(73) Assignee: Paragon 28, Inc., Englewood, CO (US)

(21) Appl. No.: 19/431,083

(22) Filed: Dec. 23, 2025

Related U.S. Application Data
(60) Provisional application No. 63/739,040, filed on Dec. 26, 2024.

(51) Int. Cl. A61B 17/64; A61B 17/00

(52) U.S. Cl. CPC A61B 17/6458; A61B 17/6425; A61B 2017/00367; A61B 2017/00477

(57) ABSTRACT

An external fixation system that includes at least one structural member and a clamp. The clamp has a bolt and an actuator, which includes a cannulation extending therethrough and a threading positioned on an inner wall of the actuator. The clamp also has a first retention component which has an upper component. The upper component includes a first upper jaw having a curved surface, a first lower jaw having a curved surface, and a first volume defined at least partially by the curved surfaces of the first upper and lower jaws and configured to receive at least a portion of the at least one structural member therein. The upper component also includes a second upper jaw having a curved surface, a second lower jaw having a curved surface, and a second volume defined at least partially by the curved surfaces of the second upper and lower jaws.

### CROSS REFERENCE TO RELATED APPLICATION

This application claims priority benefit under 35 U.S.C. § 119(e) of U.S. Provisional Application No. 63/739,040 filed Dec. 26, 2024 entitled External Fixation System, Instrumentation, and Surgical Methods (Attorney Docket No. 3645.632P1), which is incorporated herein by reference in its entirety.

### TECHNICAL FIELD

The present disclosure relates to systems, instruments, and surgical methods to be implemented in procedures and/or to address orthopedic conditions that are related to podiatric and orthopedic implants, instruments, fixation systems, and surgical methods used in various procedures and/or to address conditions of the foot, ankle, or other human anatomy. More specifically, but not exclusively, the present disclosure relates to an external fixation system, instruments, and associated methodology to be implemented in surgical procedures and/or to address conditions of the lower extremity, namely the foot and/or ankle.

### BACKGROUND OF THE INVENTION

Many currently available external fixation systems, instruments, and methods do not completely address the needs of patients. Additionally, many currently available surgical implants, instruments, and methods fail to account for properties of foot and ankle anatomy and accordingly can decrease favorability of the outcome for the patient.

### SUMMARY OF THE INVENTION

The present disclosure is directed toward systems, instruments, and surgical methods to be implemented in surgical procedures of the foot and ankle.

A first aspect of the present disclosure is an external fixation system. The external fixation system includes at least one structural member and a clamp, with the clamp including a first retention component and a second retention component.

According to the first aspect of the present disclosure, the clamp comprises a cannulation extending therethrough.

According to the first aspect of the present disclosure, the first and second retention components include central openings which are coaxial with the cannulation.

According to the first aspect of the present disclosure, the first retention element includes a first upper jaw having a curved surface, a first lower jaw having a curved surface which faces the curved surface of the first upper jaw, and a first volume defined at least partially by the curved surfaces of the first upper and lower jaws, the first volume configured to receive at least a portion of the at least one structural member therein

According to the first aspect of the present disclosure, the first retention component includes a second upper jaw having a curved surface, a second lower jaw having a curved surface which faces the curved surface of the second upper jaw, and a second volume defined at least partially by the curved surfaces of the second upper and lower jaws, the first volume configured to receive at least a portion of the at least one structural member therein.

According to the first aspect of the present disclosure, wherein the first upper jaw and second upper jaw are integral with one another.

According to the first aspect of the present disclosure, the cannulation extends through a central opening of the first retention component.

According to the first aspect of the present disclosure, the first and second volumes are positioned outside the footprint of the cannulation of the first retention component.

According to the first aspect of the present disclosure, the clamp includes an actuator. The actuator includes a threaded element extending at least partially along an axis of the cannulation of the clamp, wherein the cannulation is at least partially threaded and is coaxial with a central opening of the first retention component, an engagement feature having a threading and threadably engaged with the threaded element, and a resilient element positioned proud relative to the threaded element.

According to the first aspect of the present disclosure, the resilient element includes a spring having a longitudinal axis which is coaxial with a longitudinal axis of at least one of the cannulation and the threaded element.

A second aspect of the present disclosure is directed to an external fixation system. The system includes at least one structural member, and a clamp. The clamp includes a first retention component having a first upper jaw having a curved surface, a first lower jaw having a curved surface which faces the curved surface of the first upper jaw, and a first volume defined at least partially by the curved surfaces of the first upper and lower jaws, the first volume configured to receive at least a portion of the at least one structural member therein. The first retention element also includes a second upper jaw having a curved surface, a second lower jaw having a curved surface which faces the curved surface of the second upper jaw, and a second volume defined at least partially by the curved surfaces of the second upper and lower jaws. The clamp also includes an actuator having a threaded element extending at least partially along an axis of the cannulation of the clamp, wherein the cannulation is at least partially threaded and is coaxial with a central opening of the first retention component, an engagement feature having a threading and threadably engaged with the threaded element, and a resilient element extending at least partially around and along the threaded element.

According to the second aspect of the external fixation system, the clamp is actuatable between a first adjusting position and a second clamping position.

According to the second aspect of the external fixation system, actuating the clamp between the adjusting position and the clamping position includes manipulating the engagement feature along the threading of the threaded element such that the engagement feature is repositioned along the threading from a first position to a second position, wherein the second position is closer to the first and second upper jaws than the first position.

According to the second aspect of the external fixation system, the first adjusting position includes the engagement feature spaced from the first and second upper jaws such that either the first upper and lower jaws may be manipulated toward one another to open the second volume, or the second upper and lower jaws may be manipulated toward one another to open the first volume.

According to the second aspect of the external fixation system, the second clamping position includes the engagement feature adjacent to or in contact with the upper surface of the first and second upper jaws such that the first and second volumes remain constant despite pressure on the first upper and lower jaws or the second upper and lower jaws.

According to the second aspect of the external fixation system, the clamp includes the at least one structural member at least partially disposed within the first or second volume in the second clamping position. A third aspect of the present disclosure is an external fixation system. The system includes at least one structural member, and a clamp. The clamp includes a bolt having a head and a threaded shaft extending from the head. The clamp also includes an actuator having a cannulation extending therethrough and a threading positioned on an inner wall of the actuator which defines the cannulation, wherein the threading is complimentary to the threaded shaft. The system also includes a first retention component, which includes an upper component. The upper component includes a first upper jaw having a curved surface, a first lower jaw having a curved surface which faces the curved surface of the first upper jaw, and a first volume defined at least partially by the curved surfaces of the first upper and lower jaws, the first volume configured to receive at least a portion of the at least one structural member therein. The clamp also includes a second upper jaw having a curved surface, a second lower jaw having a curved surface which faces the curved surface of the second upper jaw, and a second volume defined at least partially by the curved surfaces of the second upper and lower jaws.

According to the third aspect of the present disclosure, the first upper jaw is integral with the second upper jaw, and the first lower jaw is integral with the second lower jaw

According to the third aspect of the present disclosure, the external fixation system also includes a second retention element. The second retention element includes a first upper jaw comprising a curved surface, a first lower jaw comprising a curved surface which faces the curved surface of the first upper jaw, and a first volume defined at least partially by the curved surfaces of the first upper and lower jaws, the first volume configured to receive at least a portion of the at least one structural member therein. The second retention element also includes a second upper jaw comprising a curved surface, a second lower jaw comprising a curved surface which faces the curved surface of the second upper jaw, and a second volume defined at least partially by the curved surfaces of the second upper and lower jaws

According to the third aspect of the present disclosure, the first and second retention members include openings that are coaxial to one another.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a front, left, perspective view of an exemplary orthopedic external fixation system, in accordance with the present disclosure;

FIG. 2 is a front, right, perspective view of the exemplary orthopedic external fixation system of FIG. 1, in accordance with the present disclosure;

FIG. 3 is a front view of the exemplary orthopedic external fixation system of FIG. 1, in accordance with the present disclosure;

FIG. 4 is a rear view of the exemplary orthopedic external fixation system of FIG. 1, in accordance with the present disclosure;

FIG. 5 is a top view of the exemplary orthopedic external fixation system of FIG. 1, in accordance with the present disclosure;

FIG. 6 is a bottom view of the exemplary orthopedic external fixation system of FIG. 1, in accordance with the present disclosure;

FIG. 7 is a right, side view of the exemplary orthopedic external fixation system of FIG. 1, in accordance with the present disclosure;

FIG. 8 is a left, side view of the exemplary orthopedic external fixation system of FIG. 1, in accordance with the present disclosure;

FIG. 9 is a front, right, perspective view of an exemplary clamp device which may be implemented in conjunction with the exemplary orthopedic external fixation system of FIG. 1, in accordance with the present disclosure;

FIG. 10 is a rear, left, perspective view of the exemplary clamp device of FIG. 9 which may be implemented in conjunction with the exemplary orthopedic external fixation system of FIG. 1, in accordance with the present disclosure;

FIG. 11 is a front view of the exemplary clamp device of FIG. 9 which may be implemented in conjunction with the exemplary orthopedic external fixation system of FIG. 1, in accordance with the present disclosure;

FIG. 12 is a rear view of the exemplary clamp device of FIG. 9 which may be implemented in conjunction with the exemplary orthopedic external fixation system of FIG. 1, in accordance with the present disclosure;

FIG. 13 is a top view of the exemplary clamp device of FIG. 9 which may be implemented in conjunction with the exemplary orthopedic external fixation system of FIG. 1, in accordance with the present disclosure;

FIG. 14 is a bottom view of the exemplary clamp device of FIG. 9 which may be implemented in conjunction with the exemplary orthopedic external fixation system of FIG. 1, in accordance with the present disclosure;

FIG. 15 is a left, side view of the exemplary clamp device of FIG. 9 which may be implemented in conjunction with the exemplary orthopedic external fixation system of FIG. 1, in accordance with the present disclosure;

FIG. 16 is a right, side view of the exemplary clamp device of FIG. 9 which may be implemented in conjunction with the exemplary orthopedic external fixation system of FIG. 1, in accordance with the present disclosure;

FIG. 17 is a right, cross-section side view of the exemplary clamp device of FIG. 9 which may be implemented in conjunction with the exemplary orthopedic external fixation system of FIG. 1, in accordance with the present disclosure

FIG. 18 is an alternate left, side view of the exemplary clamp device of FIG. 9 which may be implemented in conjunction with the exemplary orthopedic external fixation system of FIG. 1, in accordance with the present disclosure;

FIG. 19 is an alternate right, side view of the exemplary clamp device of FIG. 9 which may be implemented in conjunction with the exemplary orthopedic external fixation system of FIG. 1, in accordance with the present disclosure; and

FIG. 20 is a top, perspective view of the exemplary clamp device of FIG. 9 which may be implemented in conjunction with the exemplary orthopedic external fixation system of FIG. 1, in accordance with the present disclosure.

### DETAILED DESCRIPTION OF THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

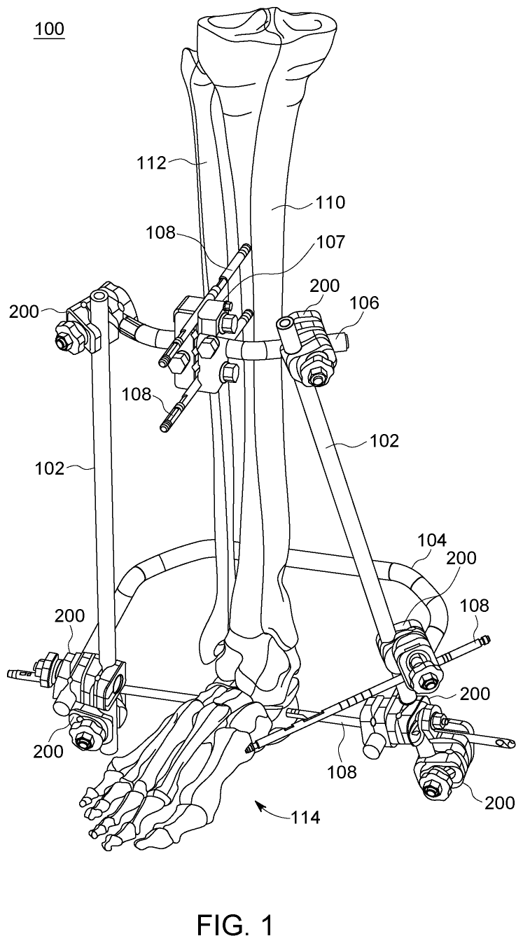

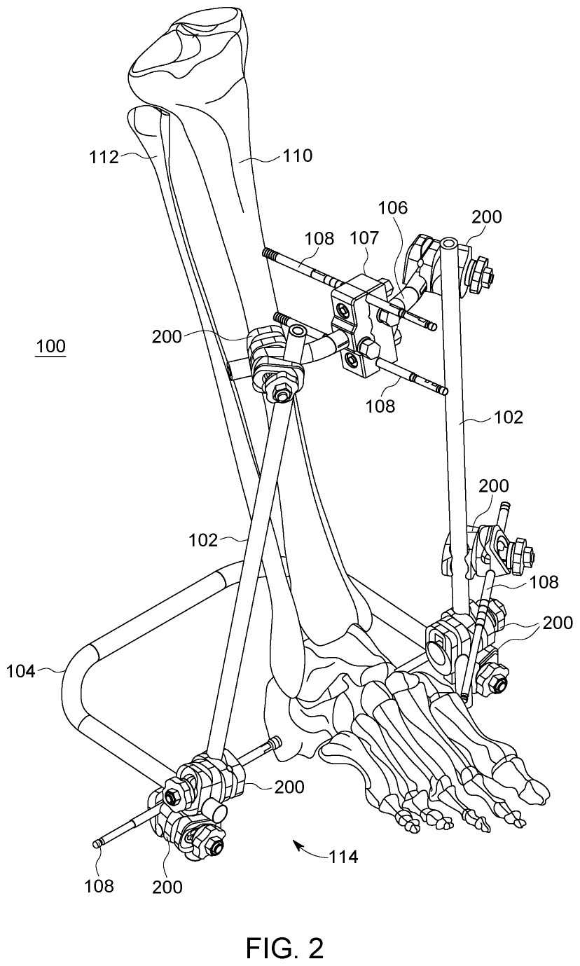

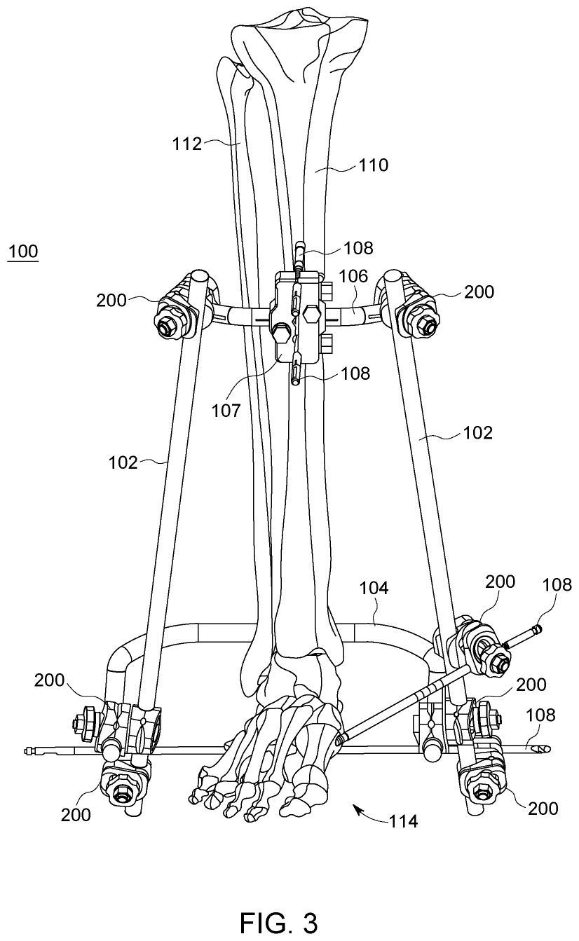

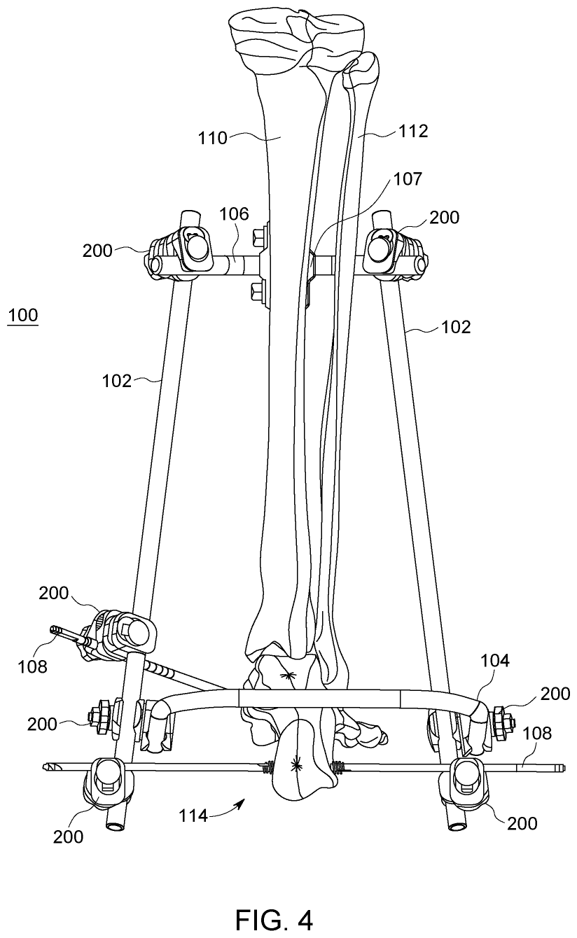

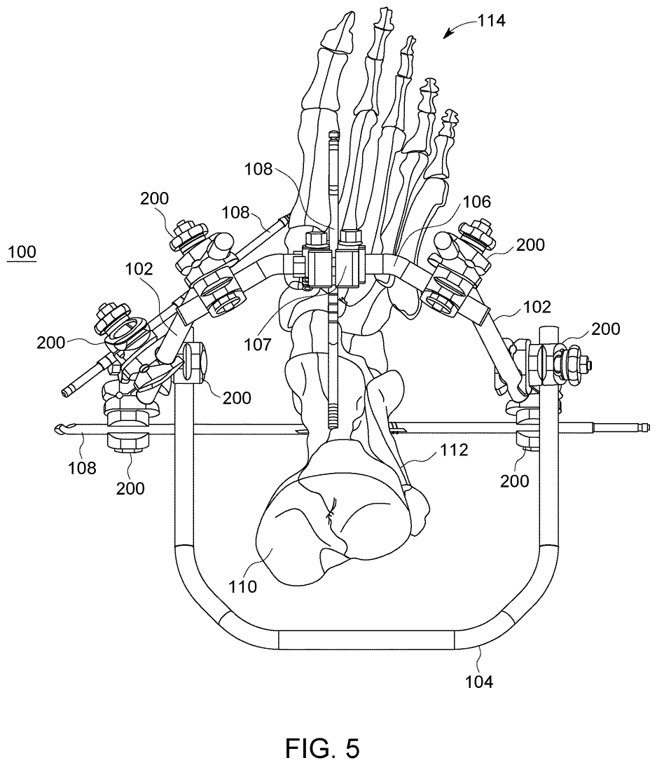

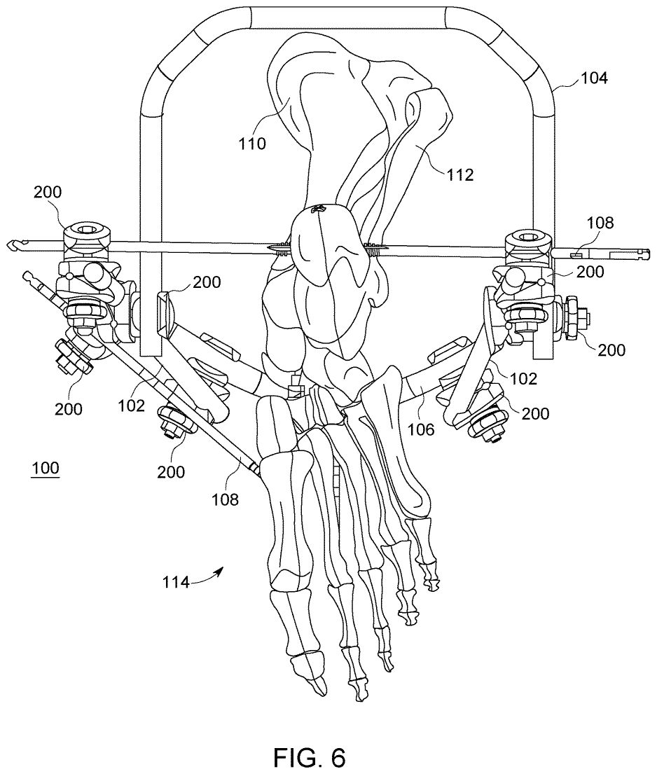

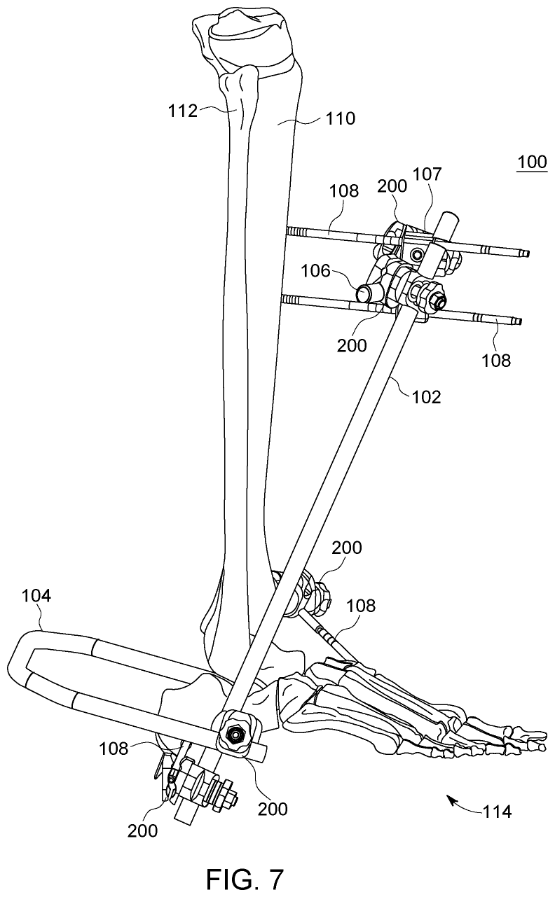

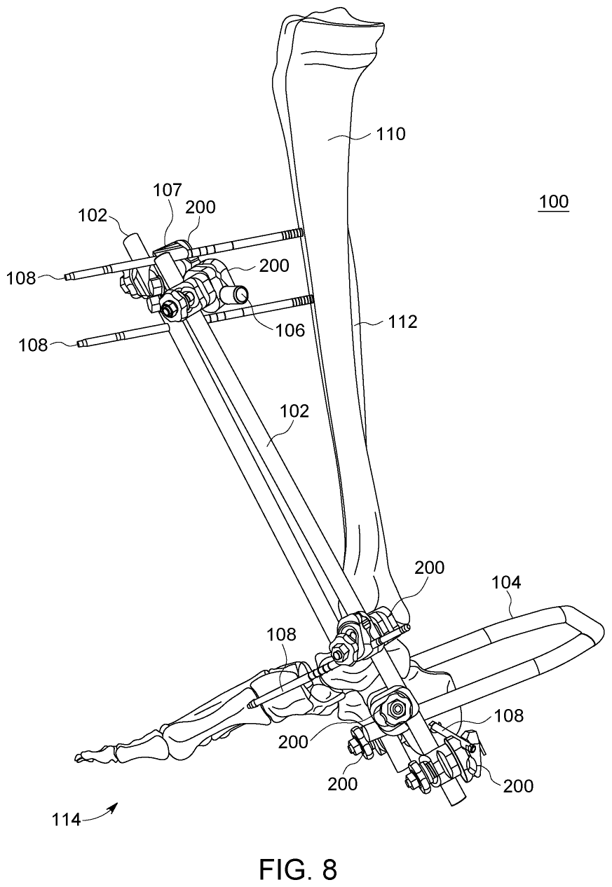

Referring now to FIGS. 1-8, an orthopedic implant system 100 and components thereof are shown, according to an exemplary embodiment. The system 100 and the components thereof, which may include one or more implants and/or instruments commonly implemented in orthopedic surgical procedures, may be implemented according to one or more various surgical methods. It should be understood that, in some aspects, the system 100 may include duplicates of one or more components, wherein one or multiple of the components are implemented in conjunction with the system 100. Further, it should be understood that in implementing the system 100, one or more of the components of the system 100 with reference to the exemplary embodiments shown and described herein may be omitted.

The system 100, as shown in the exemplary embodiment of FIGS. 1-8, is an orthopedic external fixation system. In some aspects, the system 100 may be implemented to address one or more injuries, deformities, or other conditions of a lower extremity of a patient, for example including anatomical structures such as the tibia, fibula, calcaneus, talus, navicular, and other adjacent anatomy. However, in some aspects one or more components of the system 100 may be implemented as part of an orthopedic external fixation system used to address one or more injuries, deformities, or other conditions of upper extremities or anatomy of a patient other than that of the lower extremities. Further, the system 100 may be implemented in various configurations to address different anatomy and/or conditions of a given patient. Accordingly, the configurations shown and described herein are exemplary and not in any way limiting as to the possible configurations of the system 100.

The system 100 is shown to include a pair of linear members 102 and at least one curved member 104, with each of the linear members 102 releasably coupled with the curved member 104 at opposing ends thereof via a clamp 200, as shown in FIGS. 1-8. The curved member 104 is shown to have a substantially U-shaped geometry. As shown, the curved member 104 is positioned such that it extends from a medial side of the lower extremity and around the posterior portion of the lower extremity to the lateral side of the lower extremity, with the linear members 102 coupled with ends of the curved member 104 at medial and lateral locations relative to the lower extremity. The system 100 is further shown to include at least one angled member 106, which is releasably coupled with each of the linear members 102 via clamps 200 (and, as shown in the exemplary embodiment of FIGS. 1-8, different clamps than those releasably coupling the linear members 102 with the curved member 104). The angled member 106, as shown, includes a linear portion positioned between two end portions extending from opposite ends of the linear portion at oblique angles therefrom. In some aspects, the end portions may be shorter than the linear portion of the angled member 106. As shown, the linear members 102, the curved member 104, and the angled member 106 and are substantially cylindrical. In some aspects, the linear members 102, the curved member 104, and/or the angled member 106 may be aluminum, stainless steel, carbon fiber, or other compositions and may be available in solid or hollow configurations. In some aspects, the linear members 102, the curved member 104, and/or the angled member 106 may be radiolucent or radiopaque.

The system 100 is further shown to include a plurality of pins 108, positioned variously about the system 100 as constructed in the exemplary embodiment shown in FIGS. 1-8. The pins 108 are shown to be releasably couplable at a first end with one or more components of the system 100 including, for example, the clamps 200, and releasably couplable at a second end with one or more aspects of a patient's anatomy (e.g., a tibia 110, a fibula 112, or other bony anatomy including but not limited to a calcaneus, talus, or other anatomy of a foot 114, ankle, or lower extremity). The pins 108 are shown to have a substantially cylindrical geometry with a lesser lateral dimension than that of the linear members 102, the curved member 104, and the angled member 106. In some aspects, the pins 108 may include a coupling feature at the second end configured to facilitate fixation in and coupling with bony anatomy (e.g., the tibia 110, fibula 112). Such a coupling feature may include, for example, a threading and/or cutting flutes which, when implemented in conjunction with a powered instrument (e.g., a driver, etc.) may facilitate driving the pin 108 into the aforementioned bony anatomy of the patient.

The system 100 is further shown to include a coupling 107 which, as shown in the exemplary embodiment of FIGS. 1-8, is configured to couple with one or more pins 108 and/or the angled member 106 (or other substantially cylindrically-shaped members of similar lateral dimension). The coupling 107 is shown to include one or more first through holes extending therethrough the coupling 107 and configured to receive at least a portion of a pin 108 at least partially therein and therethrough. In some aspects, such first through holes may be positioned such that longitudinal axes thereof are substantially parallel to one another. The coupling 107 may also include a second through hole having a longitudinal axis positioned in a plane substantially perpendicular to that of the first through holes, with the second through hole configured to receive at least a portion of a component with a substantially cylindrical geometry along at least a portion thereof including, for example, the linear members 102, the curved member 104, and/or the angled member 106. In some aspects, the coupling 107 may include one or more actuators that, when manipulated, may adjust the dimensions of the first and/or second through holes so as to facilitate positioning of one or elements at least partially therein/therethrough.

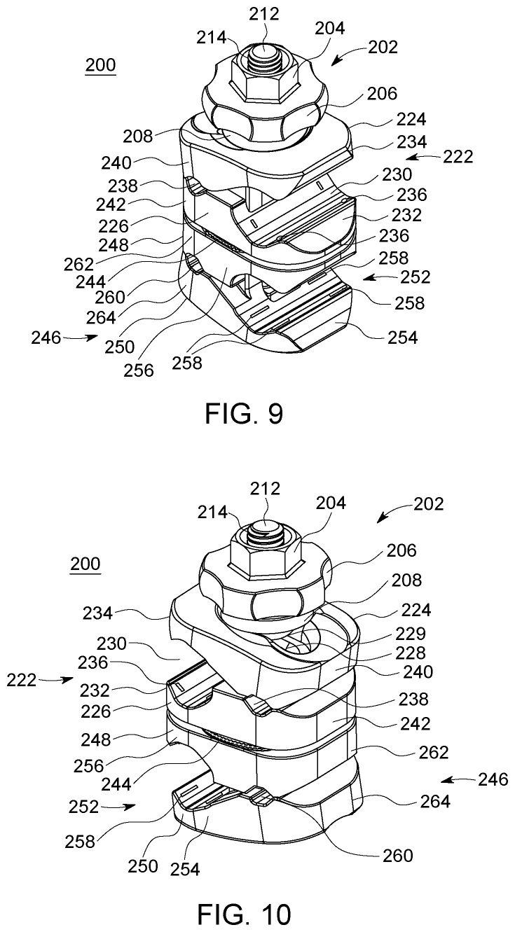

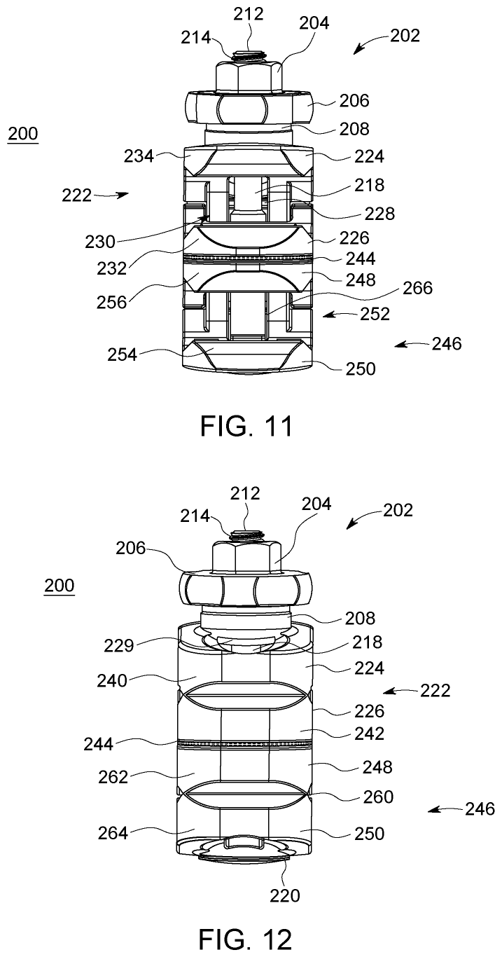

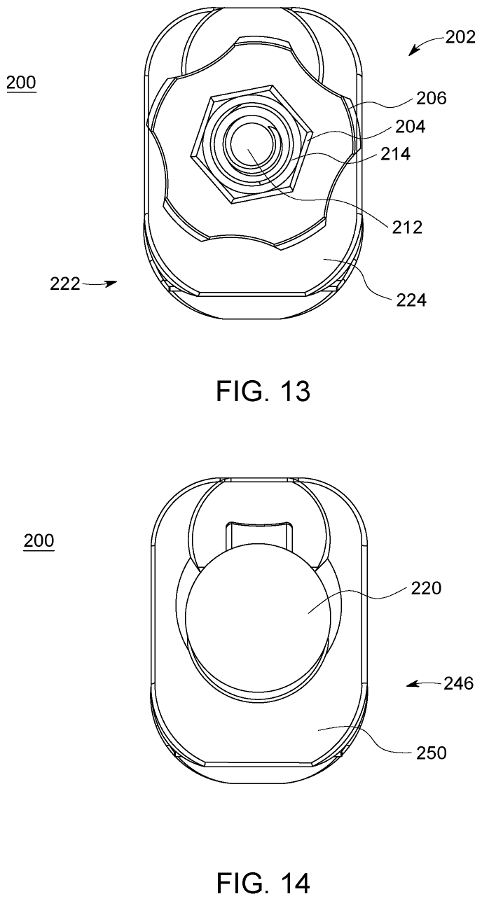

Referring now to FIGS. 9-17, the clamp 200 is shown, according to an exemplary embodiment. As described with reference to FIGS. 1-8, the system 100 may be implemented with multiple clamps 200 so as to facilitate coupling and positioning of various elements of the system 100 including but not limited to those shown and described herein. In some aspects, one or more features of the clamp 200 may be adjustable using standard tools or instruments commonly found in healthcare facilities and implemented in surgical procedures.

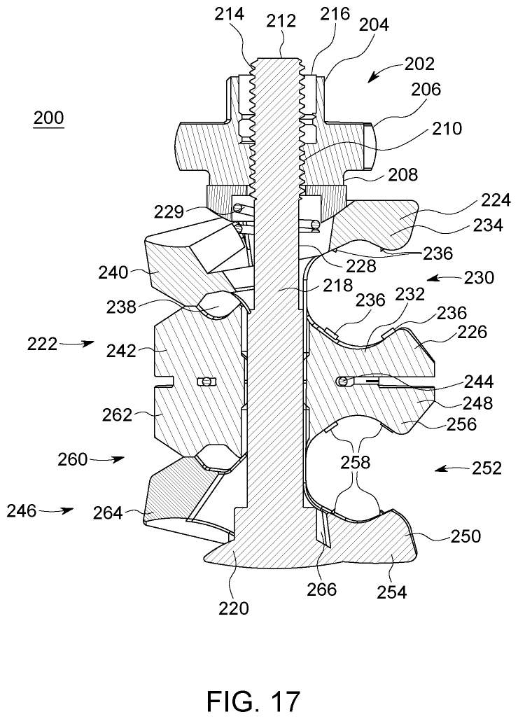

The clamp 200 is seen to include a top portion 208 positioned in the orientation of the exemplary embodiment in FIGS. 9-17, at an upper portion of the clamp 200. As seen in at least FIGS. 1-8, one or more clamps 200 may be implemented in various configurations and orientations in conjunction with the system 100. The top portion 208 is shown to include an actuator 202, which includes a hex feature 204 and an engagement feature 206 disposed thereon. In some aspects, the hex feature 204 may be configured to facilitate actuation (e.g., tightening, loosening, rotating, etc.) of the actuator 202 using an instrument with a corresponding or complimentary interface/geometry to that of the hex feature 204. Similarly, the engagement feature 206 may also be configured to facilitate actuation of the actuator 202 by a user. For example, the engagement feature 206 may include various ridges, convexities, and/or concavities configured to facilitate gripping and hand-tightening by a user. As shown in at least FIG. 17, the actuator 202 is shown to include a cannulation 216 extending along a longitudinal axis of the actuator 202. The actuator 202 includes a threading 210 extending at least partially around the inner surface of the actuator 202 which defines the cannulation 216. Such threading 210 may be sized and configured to interface with a complimentary threading of another component of the clamp 200. In some aspects, actuation of the actuator 202 via one or both of the hex feature 204 and the engagement feature 206 may facilitate translation of the actuator 202 about a threading of another component (e.g., a threaded rod, bolt, etc.).

The clamp 200 is further shown to include a bolt 212 which has a head 220 and a shaft 218, with the shaft 218 extending from the head 220, according to the exemplary embodiment of at least FIGS. 9-17. The shaft 218 has a substantially cylindrical geometry and a lateral dimension that is lesser than the lateral dimension of the head 220. The shaft 218 is shown to include a threading 214 disposed along at least a portion of the length of the shaft 218. As shown in at least FIG. 17, the threading 214 of the shaft 218 may be configured to interface with the threading 210 of the actuator 202. Accordingly, manipulation of the hex feature 204 and/or the engagement feature 206 of the actuator may advance the threading 210 along the threading 214 and thus translate the actuator 202 along at least a portion of the threading 214 (and thus, the shaft 210).

The clamp 200 is shown to include a first retention portion 222 (referred to hereinafter as “first portion 222”), according to an exemplary embodiment. The first portion 222 is shown to include a cannulation 228 extending through the first portion 222. The cannulation 228 is a substantially cylindrical volume with a lateral dimension such that the shaft 218 of the bolt 212 may be received at least partially therein and therethrough the cannulation 228. The clamp 200 is also shown to include a resilient member 229 (e.g., a spring) housed within at least a portion of the cannulation 228 of the first portion 222. In some aspects, the resilient member 229 may be housed partially within the cannulation 228 of the first portion 222 and partially within and/or in an opening adjacent to the cannulation 216 of the actuator 202. As shown in FIG. 17, the resilient member 229 may be positioned such that the shaft 218 of the bolt 212 extends through a central portion of the resilient member 229, with the resilient member 229 extending about/around the outer surface of the shaft 218. The resilient member 226 may be compressed between a portion of the first portion 222 and a portion of the actuator 202 as the actuator 202 is translated along the threading 214 of the shaft 218.

The first portion 222 is shown to include an upper component 224 and a lower component 226, according to an exemplary embodiment. As shown in at least FIG. 17, the cannulation 228 extends through both the upper and lower components 224, 226 such that the openings in the upper and lower components 224, 226 which define the cannulation 228 are coaxial. Accordingly, the shaft 218 of the bolt 212 may be at least partially received therein and therethrough the upper and lower components 224, 226 along the cannulation 228. The upper and lower components 224, 226 are thus translatable and pivotable/rotatable about the shaft 218 of the bolt 212.

The upper component 222 is shown to include a first upper jaw 234 positioned opposite the upper component 222 (e.g., opposite the opening thereof defining the cannulation 228) from a second upper jaw 240. Similarly, the lower component 224 is shown to include a first lower jaw 232 positioned opposite the lower component 224 (e.g., opposite the opening thereof defining the cannulation 228) from a second lower jaw 242. The first upper and lower jaws 234, 232 are positioned above/below one another such that the first upper and lower jaws 234, 232 are in the same rotational position about the shaft 218 of the bolt 212. The first upper jaw 234 includes a concavity defining a curved surface thereof, with the curved surface positioned such that it is facing the first lower jaw 232. The curved surface of the first upper jaw 234 defines, in part, a volume 230 positioned between the curved surface and the first lower jaw 232. FIGS. 9-17 show the curved surface of the first upper jaw 234 being directed in a downward direction (e.g., toward the first lower jaw 232).

The first lower jaw 232 is also shown to include a concavity defining a curved surface thereof, with the curved surface positioned such that it is facing the curved surface of the first upper jaw 234. The curved surface of the first lower jaw 232 defines, in part, the volume positioned between the curved surface and the curved surface of the first upper jaw 234. As shown in at least FIGS. 9-17, the curved surface of the first lower jaw 232 is directed in a downward direction (e.g., toward the first upper jaw 234 and the volume 230). The curved surface of the first upper and lower jaws 234, 232 include at least one protrusion 236 disposed on and extending upward from the respective curved surfaces. As shown with respect to the exemplary embodiment of the clamp 200, each curved surface of the first upper and lower jaws 234, 232 is shown to include a pair of protrusions 236 positioned adjacent to the terminal ends of each of the first upper and lower jaws 234, 232 and extending toward/into the volume 230.

The volume 230 positioned between the first upper and lower jaws 234, 232 is shown to be defined about a majority of the perimeter of the volume by the curved surfaces of the first upper and lower jaws 234, 232. A portion of the volume 230 is undefined and disposed between the terminal ends of the first upper and lower jaws 234, 232. This undefined portion of the volume (and thus, the positioning of at least one of the first upper and lower jaws 234, 232) may be adjustable in dimension. The first portion 222 (and thus, at least one of the first upper and lower jaws 234, 232) may be biased by a user in a direction opposite the opposing jaw so as to increase the distance between the terminal ends of the first upper and lower jaws 234, 232, and thus increasing the size of the undefined portion of the volume 230. For example, as the first upper jaw 234 is biased upward and away from the first lower jaw 232, the undefined portion of the volume 230 increases. Simultaneously, the resilient member 229 may be compressed and, when the upward biasing force applied to the first upper jaw is stopped, the resilient member 229 biases the first upper jaw 234 back to its original position. By increasing the distance between the terminal ends of the first upper and lower jaws 234, 232, a user may insert a portion of a linear member 102, a curved member 104, or an angled member 106 at least partially within the volume 230. For example, a linear member 202 may be pushed against the terminal ends of the first upper and/or lower jaws 234, 232 so as to bias the first upper jaw 234 upward, thus accepting the linear member 102 between the terminal ends of the first upper and lower jaws 234, 232 and into the volume 230. Once positioned within the volume 230, the linear member may be retained positionally (e.g., restricted from translating/rotating within the volume 230) by the protrusions 236.

It should be understood that, in some aspects the actuator 202 may be positioned adjacent the first upper component 224 so as to restrict upward movement of the first upper jaw 234 and, accordingly, restrict any increasing of the distance between the terminal ends of the first upper and lower jaws 234, 232. A user may actuate the actuator 202 in an upward direction (e.g., opposite that from the first upper component 224) such that the actuator 202 does not mechanically prohibit upward movement of the first upper jaw 234 (and thus increasing of the distance between terminal ends of the first upper and lower jaws 234, 232) so as to permit placement of a linear member 102 within the volume 230 or facilitate removal of a linear member 102 from the volume 230. Accordingly, the cannulation 228 has a lateral dimension that is greater than that of the shaft 218 of the bolt 212 such that at least one of the first upper and/or lower jaws 234, 232 may be manipulated at an oblique angle relative to the longitudinal axis of the cannulation 228 without immediate mechanical interference from the shaft 218 or the walls of the first upper or lower component 224, 226 which define the cannulation 228.

The first upper and lower components 224, 226 are shown to include a second upper jaw 240 and a second lower jaw 242, respectively. FIG. 17 shows the second upper and lower jaws 240, 242 being positioned diametrically opposite (and at an opposite end of the first upper and lower components 224, 226) the cannulation 228 from the first upper and lower jaws 234, 232, respectively. As shown in FIGS. 9-17, the second upper and lower jaws 240, 242 have a shorter distance between the longitudinal axis of the cannulation 228 and the terminal ends of the second upper and lower jaws 240, 242. Similar to the first upper and lower jaws 234, 232 the second upper and lower jaws 240, 242 are each shown to include concave, curved surfaces facing one another (with the curved surface of the second upper jaw 240 facing downward toward the second lower jaw 242, and the curved surface of the second lower jaw 242 facing upward toward the second upper jaw 240). Accordingly, a volume 238 is defined at least partially by the second upper and lower jaws 240, 242 and the curved surfaces thereof. As shown in the exemplary embodiment of FIGS. 9-17, the volume 238 is a lesser volume than the volume 230. Similar to the first upper and lower jaws 234, 232, the second upper and lower jaws 240, 242 may be manipulated by applying a force to at least one of the second upper and/or lower jaws 240, 242 to create or increase distance between terminal ends of the second upper and lower jaws 240, 242. Accordingly, a user may apply a biasing force to one or both of the second upper and lower jaws 240, 242 so as to create a distance between the terminal ends thereof and thus permit placement of a substantially cylindrical, elongated element (e.g., a pin 108 or other similar structural element that may be implemented in conjunction with the system 100) within the volume 238.

Similar to the first and second upper jaws 234, 232, the position of the actuator 202 along the shaft 218 of the bolt 212 may mechanically restrict or permit movement of the second upper and/or lower jaws 240, 242 so as to create or increase a distance between the terminal ends thereof. For example, if the actuator 202 is positioned adjacent the second upper jaw 240, a user may be required to translate the actuator along the shaft 218 via the threading 214 thereof so as to mechanically permit upward movement of the second upper jaw 240 to thus permit placement of a structural member within the volume 238. Accordingly, such upward movement of the second upper jaw 240 may be resisted by the resilient member 239. Once the structural member is placed within the volume 238, a user may stop the application of any upward force to the second upper and/or lower jaws 240, 242 and translate the actuator 202 to its original position abutting the second upper jaw 240 so as to retain the structural member within the volume 238.

In some aspects, one or both of the upper and lower components 224, 226 may include a protrusion or other point disposed between the curvatures between the first and second upper jaws 234, 240 and the first and second lower jaws 232, 242 extending upward toward the opposite component (either the upper or lower component 224, 226). Such protrusion or point may be a fulcrum point or “rocker” and facilitate the application of a force to, and the creation or increase of a distance between, at least one of the first upper or lower jaws 234, 232 or at least one of the second upper or lower jaws 240, 242. Similarly, the first portion 222 may include a cavity 266 extending into one or both of the upper and lower components 224, 226 configured to receive at least a portion of the shaft 218 to facilitate manipulation of the first and/or second component 224, 226 to facilitate placement of a structural member within the volume 230 or 238.

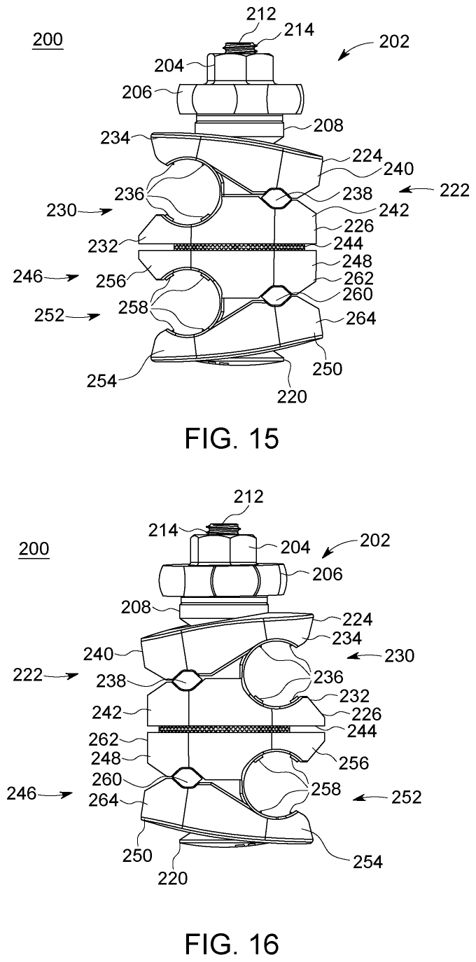

The clamp 200 is also shown to include a second retention portion 246 (referred to hereinafter as “second portion 246”), which is positioned below the first portion 222. As shown in at least FIGS. 15-17, the clamp 200 is also shown to include a rotation member 244 disposed between the first and second portions 222, 246. The rotation member 244 may include a central opening defining the cannulation 228 which extends therethrough and may also receive at least a portion of the shaft 218 of the bolt 212 therein and therethrough the central opening. The rotation member 244, as shown in the exemplary embodiment of FIGS. 9-17, is configured to facilitate rotation of the first portion 222 relative to the second portion 246 (or vice-versa) about the shaft 218 of the bolt 212. For example, the first portion 222 may be rotated 90 degrees about the shaft 218 to facilitate coupling with structural members of the system 100 in various different positions and/or planes.

The second portion 246 is shown to include the cannulation 228 extending therethrough the second portion 246. As shown, the cannulation 228 is a substantially cylindrical volume with a lateral dimension such that the shaft 218 of the bolt 212 may be received at least partially therein and therethrough the cannulation 228. The second portion 246 is also shown to include an upper component 248 and a lower component 250, according to an exemplary embodiment. As shown in at least FIG. 17, the cannulation 228 extends through both the upper and lower components 248, 250 such that the openings in the upper and lower components 248, 250 which define the cannulation 228 are coaxial. Accordingly, the shaft 218 of the bolt 212 may be at least partially received therein and therethrough the upper and lower components 248, 250 along the cannulation 228. The upper and lower components 248, 250 are translatable and pivotable/rotatable about the shaft 218 of the bolt 212.

The upper component 222 is shown to include a first upper jaw 256 positioned opposite the upper component 222 (e.g., opposite the opening thereof defining the cannulation 228) from a second upper jaw 262. Similarly, the lower component 248 is shown to include a first lower jaw 254 positioned opposite the lower component 248 (e.g., opposite the opening thereof defining the cannulation 228) from a second lower jaw 264. The first upper and lower jaws 256, 254 are shown to be positioned above/below one another such that the first upper and lower jaws 256, 254 are in the same rotational position about the shaft 218 of the bolt 212. The first upper jaw 256 is shown to include a concavity defining a curved surface thereof, with the curved surface positioned such that it is facing the first lower jaw 254. The curved surface of the first upper jaw 256 defines, in part, a volume 252 positioned between the curved surface and the first lower jaw 254. As shown in at least FIGS. 9-17, the curved surface of the first upper jaw 256 is directed in a downward direction (e.g., toward the first lower jaw 254).

The first lower jaw 254 is also shown to include a concavity defining a curved surface thereof, with the curved surface positioned such that it is facing the curved surface of the first upper jaw 256. The curved surface of the first lower jaw 254 defines, in part, the volume positioned between the curved surface and the curved surface of the first upper jaw 256. As shown in at least FIGS. 9-17, the curved surface of the first lower jaw 254 is directed in a downward direction (e.g., toward the first upper jaw 256 and the volume 252). The curved surface of the first upper and lower jaws 256, 254 are shown to include at least one protrusion 258 disposed on and extending upward from the respective curved surfaces. As shown with respect to the exemplary embodiment of the clamp 200, each curved surface of the first upper and lower jaws 256, 254 is shown to include a pair of protrusions 258 positioned adjacent to the terminal ends of each of the first upper and lower jaws 256, 254 and extending toward/into the volume 252.

The volume 252 positioned between the first upper and lower jaws 256, 254 is shown to be defined about a majority of the perimeter of the volume by the curved surfaces of the first upper and lower jaws 256, 254. A portion of the volume 252 is undefined and disposed between the terminal ends of the first upper and lower jaws 256, 254. This undefined portion of the volume (and thus, the positioning of at least one of the first upper and lower jaws 256, 254) may be adjustable in dimension. At least the second portion 246 (and thus, at least one of the first upper and lower jaws 256, 254) may be biased by a user in a direction opposite the opposing jaw so as to increase the distance between the terminal ends of the first upper and lower jaws 256, 254, and thus increasing the size of the undefined portion of the volume 252. For example, as the first upper jaw 256 is biased upward and away from the first lower jaw 254, the undefined portion of the volume 252 increases. Simultaneously, the resilient member 229 may be compressed (via the biasing force applied to the first upper jaw 256 driving the rotation member 244 and/or first portion 222 along the shaft so as to compress the resilient member 229) and, when the upward biasing force applied to the first upper jaw 256 is stopped, the resilient member 229 biases the first upper jaw 256 back to its original position (via the first portion 222 or rotation member 244 contacting the first upper jaw 256). By increasing the distance between the terminal ends of the first upper and lower jaws 256, 254, a user may insert a portion of a linear member 102, a curved member 104, or an angled member 106 at least partially within the volume 252. For example, a linear member 202 may be pushed against the terminal ends of the first upper and/or lower jaws 256, 254 so as to bias the first upper jaw 256 upward, thus accepting the linear member 102 between the terminal ends of the first upper and lower jaws 256, 254 and into the volume 252. Once positioned within the volume 252, the linear member may be retained positionally (e.g., restricted from translating/rotating within the volume 252) by the protrusions 258.

It should be understood that, in some aspects the actuator 202 may be positioned adjacent the first upper component 224 of the first portion 222 so as to restrict upward movement of the first upper jaw 256 and, accordingly, restrict any increasing of the distance between the terminal ends of the first upper and lower jaws 256, 254. A user may actuate the actuator 202 in an upward direction (e.g., opposite that from the first upper component 224 of the first portion 222) such that the actuator 202 does not mechanically prohibit (via the first portion 222 and/or rotation member 244) upward movement of the first upper jaw 256 (and thus increasing of the distance between terminal ends of the first upper and lower jaws 256, 254) so as to permit placement of a linear member 102 within the volume 252 or facilitate removal of a linear member 102 from the volume 252. Accordingly, the cannulation 228 has a lateral dimension that is greater than that of the shaft 218 of the bolt 212 such that at least one of the first upper and/or lower jaws 256, 254 may be manipulated at an oblique angle relative to the longitudinal axis of the cannulation 228 without immediate mechanical interference from the shaft 218 or the walls of the first upper or lower component 248, 250 which define the cannulation 228.

The first upper and lower components 248, 250 are shown to include a second upper jaw 262 and a second lower jaw 264, respectively. As shown in at least FIG. 17, the second upper and lower jaws 262, 264 are positioned diametrically opposite (and at an opposite end of the first upper and lower components 248, 250) the cannulation 228 from the first upper and lower jaws 256, 254, respectively. As shown in FIGS. 9-17, the second upper and lower jaws 262, 264 have a shorter distance between the longitudinal axis of the cannulation 228 and the terminal ends of the second upper and lower jaws 262, 264. Similar to the first upper and lower jaws 256, 254 the second upper and lower jaws 262, 264 are each shown to include concave, curved surfaces facing one another (with the curved surface of the second upper jaw 262 facing downward toward the second lower jaw 264, and the curved surface of the second lower jaw 264 facing upward toward the second upper jaw 262). Accordingly, a volume 260 is defined at least partially by the second upper and lower jaws 262, 264 and the curved surfaces thereof. As shown in the exemplary embodiment of FIGS. 9-17, the volume 260 is a lesser volume than the volume 252. Similar to the first upper and lower jaws 256, 254, the second upper and lower jaws 262, 264 may be manipulated by applying a force to at least one of the second upper and/or lower jaws 262, 264 so as to create or increase distance between terminal ends of the second upper and lower jaws 262, 264. Accordingly, a user may apply a biasing force to one or both of the second upper and lower jaws 262, 264 so as to create a distance between the terminal ends thereof and thus permit placement of a substantially cylindrical, elongated element (e.g., a pin 108 or other similar structural element that may be implemented in conjunction with the system 100) within the volume 260.

Similar to the first and second upper jaws 256, 254, the position of the actuator 202 along the shaft 218 of the bolt 212 may mechanically restrict (via the first portion 222 and/or rotation member 244) or permit movement of the second upper and/or lower jaws 262, 264 so as to create or increase a distance between the terminal ends thereof. For example, if the actuator 202 is positioned adjacent the upper component 224 of the first portion 222, a user may be required to translate the actuator along the shaft 218 via the threading 214 thereof so as to mechanically permit upward movement of the second upper jaw 262 to thus permit placement of a structural member within the volume 260. Accordingly, such upward movement of the second upper jaw 262 may be resisted by the resilient member 239 (via the first portion 222 and/or rotation member 244). Once the structural member is placed within the volume 260, a user may stop the application of any upward force to the second upper and/or lower jaws 262, 264 and translate the actuator 202 to its original position abutting the upper component 224 of the first portion 222 so as to retain the structural member within the volume 260.

In some aspects, one or both of the upper and lower components 248, 250 may include a protrusion or other point disposed between the curvatures between the first and second upper jaws 256, 262 and the first and second lower jaws 254, 264 extending upward toward the opposite component (either the upper or lower component 248, 250). Such protrusion or point may be a fulcrum point or “rocker” and thus facilitate the application of a force to, and the creation or increase of a distance between, least one of the first upper or lower jaws 256, 254 or at least one of the second upper or lower jaws 262, 264. Similarly, the second portion 246 may include the cavity 266 extending into one or both of the upper and lower components 248, 250 configured to receive at least a portion of the shaft 218 to facilitate manipulation of the first and/or second component 248, 250 to facilitate placement of a structural member within the volume 252 or 260.

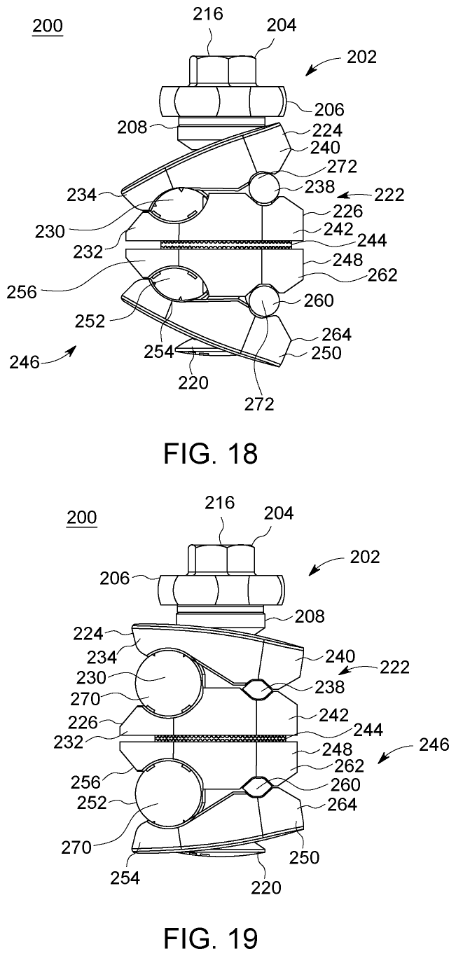

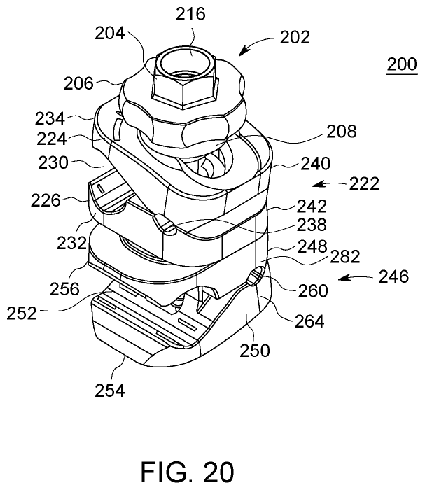

Referring now to FIGS. 18-20, the clamp 200 is shown in various configurations engaged with members of an external fixation system 100 which may be the same as and/or similar to those shown and described previously herein. The clamp 200 is shown in a first configuration in FIG. 18 in which the clamp is engaged with a cylindrical member 272 (which may be the same as or similar to the pins 108 described previously herein), which is positioned at least partially within the volume 238 (and thus between the second upper and lower jaws 240, 242 of the first retention portion 222. The second retention portion 246 of the clamp 200 is shown to be engaged with a cylindrical member 272 in a similar fashion such that the cylindrical member 272 is positioned at least partially within the volume 260 and between the second upper and lower jaws 262, 264. In some aspects, the system 100 may be configured such that the clamp 200 is engaged with one or more cylindrical members 272, with the cylindrical members 272 positioned at least partially within the volume 238 and/or 260. In some aspects, the first retention portion 222 may be rotated about a central axis of the shaft 218 of the bolt 212 relative to the second retention portion 246, for example in a position/orientation similar to that shown in the exemplary embodiment of FIG. 20. As shown in FIG. 18, the first and second retention portions 222, 246 are configured such that first lower and upper jaws 232, 234 (of first retention portion 222) and 254, 256 (of second retention portion 246) contact one another when the cylindrical members 272 are received at least partially within the volumes 238, 260, respectively.

Referring now to FIG. 19, the clamp 200 is shown in an engaged configuration with the cylindrical members 270 of the external fixation system 100 which may be the same as and/or similar to those shown and described previously herein. The clamp 200 is to be engaged with the cylindrical member 270 (which may be the same as or similar to the linear members 102, curved member 104, and/or angled member 106 as shown and described previously herein), which is positioned at least partially within the volume 230 (and thus between the first lower and upper jaws 232, 234 of the first retention portion 222. The second retention portion 246 of the clamp 200 is shown to be similarly engaged with a cylindrical member 270 in a similar fashion such that the cylindrical member 270 is positioned at least partially within the volume 252 and between the first lower and upper jaws 254, 256. In some aspects, the system 100 may be configured such that the clamp 200 is engaged with one or more cylindrical members 270 simultaneously, with the cylindrical members positioned at least partially within the volumes 230 and/or 252. In some aspects, the first retention portion 222 may be rotated about a central axis of the shaft 218 of the bolt 212 relative to the second retention portion 246, for example in a position/orientation similar to that shown in the exemplary embodiment of FIG. 20. As shown in FIG. 19, the first and second retention portions 222, 246 are configured such that second upper and lower jaws 240, 242 (of first retention portion 222) and 262, 264 (of second retention portion 246) contact one another when the cylindrical members 270 are received at least partially within the volumes 230, 252, respectively.

Engagement and/or disengagement with cylindrical members 270 and/or 272 may be accomplished in a “snap-in” and “snap-out” fashion which is enabled by the resilient member 229 of the clamp 200. For example, prior to positioning the cylindrical member 270 within the volume 230 or 252, the distance between the first lower and upper jaws 232, 234 and/or 254, 256 is lesser than a diameter of the cylindrical member 270. Accordingly, as a force is applied to the cylindrical member 270 (and thus the first upper and lower jaws 232, 234 and/or 254, 256), the distance between the first upper and lower jaws 232, 234 and/or 254, 256 may be increased as the resilient member 229 is compressed (at which point the respective second upper and lower jaws 240, 242 and/or 262, 264 contact one another), thus allowing the cylindrical element to be received within the opening 230 or 252 and retained within the opening 230, 252 by the respective first lower and upper jaws 232, 234 and/or 254, 256. Similarly, prior to positioning the cylindrical member 272 within the volume 238 or 260, the distance between the second upper and lower jaws 240, 242 and/or 262, 264 is lesser than a diameter of the cylindrical member 272. Accordingly, as a force is applied to the cylindrical element 270 (and thus the first upper and lower jaws 232, 234 and/or 254, 256), the distance between the second upper and lower jaws 240, 242 and/or 262, may be increased as the resilient member 229 is compressed (at which point the respective first lower and upper jaws 232, 234 and/or 254, 256 contact one another), thus allowing the cylindrical member 272 to be received within the opening 238 or 260 and retained within the opening 238, 260 by the respective first upper and lower jaws 232, 234 and/or 254, 256.

The aforementioned contact between the opposing jaws of a given retention portion (e.g., 222 or 256) maximizes a reaction force distance from the central axis of the shaft 218 the bolt 212 (about which each retention portion 222, 246 is pivotable/rotatable) so as to maximize clamping force. In such clamping of cylindrical members 270, 272 a “fulcrum” point (e.g., center of rotation) of the respective retention portion (e.g., 242, 246 including first lower and upper jaws 232, 234 and/or 254, 256 and second upper and lower jaws 240, 242 and/or 262, 264) is established along the longitudinal axis of the cylindrical member 270, 272.

The terminology used herein for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. An external fixation system, comprising:
at least one structural member; and
a clamp, comprising:
a first retention component; and
a second retention component.

2. The external fixation system of claim 1, wherein the clamp comprises a cannulation extending therethrough.

3. The external fixation system of claim 2, wherein the first and second retention components comprise central openings which are coaxial with the cannulation.

4. The external fixation system of claim 3, wherein the first retention component comprises:
a first upper jaw comprising a curved surface;
a first lower jaw comprising a curved surface which faces the curved surface of the first upper jaw; and
a first volume defined at least partially by the curved surfaces of the first upper and lower jaws, the first volume configured to receive at least a portion of the at least one structural member therein.

5. The external fixation system of claim 4, wherein the first retention component further comprises:
a second upper jaw comprising a curved surface;
a second lower jaw comprising a curved surface which faces the curved surface of the second upper jaw; and
a second volume defined at least partially by the curved surfaces of the second upper and lower jaws, the first volume configured to receive at least a portion of the at least one structural member therein.

6. The external fixation system of claim 5, wherein the first upper jaw and second upper jaw are integral with one another.

7. The external fixation system of claim 6, wherein the cannulation extends through a central opening of the first retention component.

8. The external fixation system of claim 7, wherein the first and second volumes are positioned outside of a footprint of the cannulation of the first retention component.

9. The external fixation system of claim 7, wherein the clamp comprises an actuator, the actuator comprising:
a threaded element extending at least partially along an axis of the cannulation of the clamp, wherein the cannulation is at least partially threaded and is coaxial with a central opening of the first retention component;
an engagement feature comprising a threading and threadably engaged with the threaded element; and
a resilient element positioned proud relative to the threaded element.

10. The external fixation system of claim 9, wherein the resilient element comprises a spring having a longitudinal axis which is coaxial with a longitudinal axis of at least one of the cannulation and the threaded element.

11. An external fixation system, comprising:
at least one structural member; and
a clamp, comprising:
a first retention component, comprising:
a first upper jaw comprising a curved surface;
a first lower jaw comprising a curved surface which faces the curved surface of the first upper jaw;
a first volume defined at least partially by the curved surfaces of the first upper and lower jaws, the first volume configured to receive at least a portion of the at least one structural member therein;
a second upper jaw comprising a curved surface;
a second lower jaw comprising a curved surface which faces the curved surface of the second upper jaw; and
a second volume defined at least partially by the curved surfaces of the second upper and lower jaws; and
an actuator, comprising:
a threaded element extending at least partially along an axis of the cannulation of the clamp, wherein the cannulation is at least partially threaded and is coaxial with a central opening of the first retention component;
an engagement feature comprising a threading and threadably engaged with the threaded element; and
a resilient element extending at least partially around and along the threaded element.

12. The external fixation system of claim 11, wherein the clamp is actuatable between a first adjusting position and a second clamping position.

13. The external fixation system of claim 12, wherein actuating the clamp between the adjusting position and the clamping position comprises manipulating the engagement feature along the threading of the threaded element such that the engagement feature is repositioned along the threading from a first position to a second position, wherein the second position is closer to the first and second upper jaws than the first position.

14. The external fixation system of claim 12, wherein the first adjusting position comprises the engagement feature spaced from the first and second upper jaws such that either the first upper and lower jaws may be manipulated toward one another to open the second volume, or the second upper and lower jaws may be manipulated toward one another to open the first volume.

15. The external fixation system of claim 14, wherein the second clamping position comprises the engagement feature adjacent to or in contact with the upper surface of the first and second upper jaws such that the first and second volumes remain constant despite pressure on the first upper and lower jaws or the second upper and lower jaws.

16. The external fixation system of claim 15, wherein the clamp comprises the at least one structural member at least partially disposed within the first or second volume in the second clamping position.

17. An external fixation system, comprising:
at least one structural member; and
a clamp, comprising:
a bolt comprising a head and a threaded shaft extending from the head;
an actuator comprising a cannulation extending therethrough and a threading positioned on an inner wall of the actuator which defines the cannulation, wherein the threading is complimentary to the threaded shaft; and
a first retention component, comprising:
an upper component, comprising:
a first upper jaw comprising a curved surface;
a first lower jaw comprising a curved surface which faces the curved surface of the first upper jaw;
a first volume defined at least partially by the curved surfaces of the first upper and lower jaws, the first volume configured to receive at least a portion of the at least one structural member therein;
a second upper jaw comprising a curved surface;
a second lower jaw comprising a curved surface which faces the curved surface of the second upper jaw; and
a second volume defined at least partially by the curved surfaces of the second upper and lower jaws.

18. The external fixation system of claim 17, wherein the first upper jaw is integral with the second upper jaw, and the first lower jaw is integral with the second lower jaw.

19. The external fixation system of claim 18, further comprising a second retention component comprising:
a first upper jaw comprising a curved surface;
a first lower jaw comprising a curved surface which faces the curved surface of the first upper jaw;
a first volume defined at least partially by the curved surfaces of the first upper and lower jaws, the first volume configured to receive at least a portion of the at least one structural member therein;
a second upper jaw comprising a curved surface;
a second lower jaw comprising a curved surface which faces the curved surface of the second upper jaw; and
a second volume defined at least partially by the curved surfaces of the second upper and lower jaws.

20. The external fixation system of claim 19, wherein the first and second retention members comprise openings that are coaxial to one another.
